(19) United States
(12) Patent Application Publication
(10) Pub. No.: US 2026/0306375 A1
(43) Pub. Date: Oct. 8, 2026

(54) HAIR TREATMENT AGENT

(71) Applicant: NIKKO CHEMICALS CO., LTD., Chuo-ko, Tokyo (JP)

(72) Inventors: Yuichiro NOMURA, Chuo-ku, Toyko (JP); Saeka NOMURA, Chuo-ku, Tokyo (JP); Shunsuke TAKAYAMA, Chuo-ku, Tokyo (JP)

(21) Appl. No.: 19/482,839

(22) PCT Filed: May 15, 2024
(86) PCT No.: PCT/JP2024/017931

§ 371 Date: Nov. 10, 2025

(30) Foreign Application Priority Data
May 16, 2023 (JP) 2023-080873

(51) Int. Cl. A61K 8/39; A61K 8/41; A61Q 5/00; A61Q 5/12

(52) U.S. Cl. CPC A61K 8/39; A61K 8/416; A61Q 5/002; A61Q 5/12

(57) ABSTRACT

A hair treatment agent imparting the effect of moistness, slipperiness, and water repellency to damaged, hydrophilic hair is provided, the hair treatment agent being formulated with a polyglycerin hydroxystearate ester.

### FIELD OF THE INVENTION

The present invention relates to a hair treatment agent, especially a hair treatment agent having the effect of imparting sufficient moistness and slipperiness to hair after rinsing and further improving water repellency thereof.

### BACKGROUND OF THE INVENTION

In recent years, hair damage has become serious, notably among young women, due to chemical treatments such as bleaching, perming, and others. It is known that hair damage is accompanied by a loss of 18-MEA (18-methyleicosanoic acid), a fatty acid that coats the surface of hair, causing an increase in friction on the surface together with hydrophilization of the surface of hair.

Rinse-out type hair treatments form stable emulsions owing to cationic surfactants and higher alcohols. As the cationic surfactants, mono long-chain alkyl quaternary ammonium salts are primarily used, and owing to their high conditioning effect, a smooth feeling is achieved when the treatments are applied to hair. However, it is impossible to impart sufficient moistness and water repellency to hair owing to these cationic surfactants alone.

For example, JP-A 2007-145769 has already developed a technology for imparting moistness or manageability to hair, by formulating a hair treatment agent using a cationic surfactant with specific components. However, the effect of improving slipperiness or water repellency of hair after drying has not been found. Note that JP-A 2007-145769 has found advantageous effects on hair by a combination of a hydroxystearate ester and an oleate ester, but formulating hair treatment agents with oleate esters leads to high hydrophilicity, making it difficult to impart water repellency to hair to which they are applied. Further, JP-A H08-048615 has found that being formulated with a component having a perfluoroalkyl group improves water repellency of hair, but it has not been found that being formulated with polyglycerin hydroxystearate esters improves water repellency.

### SUMMARY OF THE INVENTION

The problem to be solved by the present invention is to provide a hair treatment agent having the effect of imparting sufficient moistness and slipperiness to hair after rinsing and further improving water repellency thereof.

After careful consideration of the above problem, the present inventors have found that a hair treatment agent containing a polyglycerin hydroxystearate ester (component (a)) or the hair treatment agent further containing one or two or more selected from a mono (long-chain alkyl) quaternary ammonium salt, a di (long-chain alkyl) quaternary ammonium salt, and a mono (long-chain alkyl) amine (component (b)) can solve the above problem.

In other words, in one embodiment, the present invention provides a hair treatment agent formulated with a polyglycerin hydroxystearate ester.

Further, in another embodiment, the present invention provides the hair treatment agent further formulated with one or two or more selected from a mono (long-chain alkyl) quaternary ammonium salt, a di (long-chain alkyl) quaternary ammonium salt, and a mono (long-chain alkyl) amine (component (b)).

According to the present invention, the hair treatment agent can be provided with the effect of improving moistness, slipperiness, and water repellency of hair after drying by having the above formulation components, in other words, component (a) or components (a) and (b).

### EMBODIMENTS OF THE INVENTION

Exemplary embodiments of the present invention are illustrated below, but the present invention is not limited thereto. The polyglycerin hydroxystearate ester of component (a) used in the present invention is a compound in which hydroxyl groups of polyglycerin are esterified with hydroxystearic acid. An average degree of polymerization of the polyglycerin is 5 or more and preferably 10 or more, and 20 or less and preferably 15 or less from the viewpoint of attaining high moistness, slipperiness, or water repellency of hair.

In an embodiment, the polyglycerin hydroxystearate ester used in the present invention is a polyglycerin hydroxystearate ester obtained by esterification using 0.5 to 10.0 mol and preferably 3.0 mol to 7.0 mol of hydroxystearic acid relative to 1 mol of polyglycerin with an average degree of polymerization of 5 or more as above.

In an embodiment, an esterification rate of the polyglyceryl poly (hydroxystearate) used in the present invention is expressed as [(total number of esterified hydroxyl groups of polyglycerin)/(total number of hydroxyl groups of polyglycerin)]×100(%). For example, the esterification rate may be 20 to 90% and is preferably 40 to 80% from the viewpoint of attaining high moistness, slipperiness, or water repellency of hair.

Further, in an embodiment, the hair treatment agent of the present invention is a hair treatment agent formulated with the polyglycerin hydroxystearate ester in an amount of 0.1 to 5.0 mass % of a total mass of the hair treatment agent. When the formulation amount of the polyglycerin hydroxystearate ester is less than 0.1 mass %, slipperiness, water repellency, and other effects are not sufficiently exhibited. Further, a formulation amount of more than 5.0 mass % is not preferable because stickiness specific to polyglycerin polymers becomes pronounced, which worsens slipperiness. The formulation amount of the polyglycerin hydroxystearate ester is preferably 0.3 to 3.0 mass % and more preferably 0.5 to 2.0 mass % of a total mass of the hair treatment agent from the viewpoint of attaining high moistness, slipperiness, or water repellency of hair.

In an embodiment, examples of the polyglycerin hydroxystearate ester include, but are not limited to, polyglyceryl-10 decahydroxystearate, polyglyceryl-10 heptahydroxystearate, polyglyceryl-10 pentahydroxystearate, polyglyceryl-6 pentahydroxystearate, and a combination of two or more of these, or the like. Note that the numerals after the hyphens (−) indicate average degrees of polymerization of glycerin. Among these, polyglyceryl-10 pentahydroxystearate is preferable from the viewpoint of attaining good slipperiness. Examples of commercially available products of polyglyceryl-10 pentahydroxystearate include, for example, “NIKKOL Decaglyn 5-HS” (manufactured by Nikko Chemicals Co., Ltd.), and those of polyglyceryl-10 heptahydroxystearate include, for example, “NIKKOL Decaglyn 7-HS” (manufactured by Nikko Chemicals Co., Ltd.) and others.

In an embodiment, the hair treatment agent of the present invention may further be formulated with one or two or more selected from a mono (long-chain alkyl) quaternary ammonium salt, a di (long-chain alkyl) quaternary ammonium salt, and a mono (long-chain alkyl) amine (component (b)). Examples of these compounds include, but are not limited to, cetrimonium chloride, steartrimonium chloride, behentrimonium chloride, cetrimonium bromide, steartrimonium bromide, behentrimonium bromide, cetrimonium methosulfate, steartrimonium methosulfate, behentrimonium methosulfate, dicetyldimonium chloride, distearyldimonium chloride, stearamidopropyl dimethylamine, behenamidopropyl dimethylamine, and a combination of two or more of these, and others. One or two or more selected from behentrimonium chloride, steartrimonium chloride, cetrimonium chloride, stearamidopropyl dimethylamine, and behenamidopropyl dimethylamine are preferable. A formulation amount of component (b) in the hair treatment agent of the present invention can be, for example, 0.1 to 5.0 mass % and preferably 1.0 to 3.0 mass % of a total mass of the hair treatment agent from the viewpoint of attaining high moistness, slipperiness, or water repellency of hair.

A mass ratio expressed as [content of component (b)]/[content of component (a)] can be, for example, 0.1 to 40, preferably 0.5 to 30, and more preferably 1 to 20 from the viewpoint of attaining high moistness, slipperiness, or water repellency of hair.

In an embodiment, versatile components contained in emulsions such as hair treatments and others can be added to the hair treatment agent of the present invention in a range that the effect of the present invention is not impaired. Examples of such components include, but are not limited to, hydrocarbon oils, silicone oils, oils and fats, polyhydric alcohols such as propanediol, glycerin, and others, lecithin derivatives such as hydrogenated lecithin (product name: NIKKOL LECINOL S-10M PLUS, manufactured by Nikko Chemicals Co., Ltd.) and others, glycerin fatty acid esters such as glyceryl stearate (product name: NIKKOL MGS-AV, manufactured by Nikko Chemicals Co., Ltd.) and others, sorbitan fatty acid esters such as sorbitan isostearate (product name: NIKKOL SI-10RV, manufactured by Nikko Chemicals Co., Ltd.) and others, polyoxyethylene sorbitan fatty acid esters such as polysorbate 80 (product name: NIKKOL TO-10V, manufactured by Nikko Chemicals Co., Ltd.) and others, polyoxyalkylene sterols such as PEG-30 phytosterol (product name: NIKKOL BPS-30, manufactured by Nikko Chemicals Co., Ltd.) and others, polyoxyethylene alkyl ethers such as beheneth-30 (product name: NIKKOL BB-30, manufactured by Nikko Chemicals Co., Ltd.) and others, monoalkyl phosphates such as cetyl phosphate (product name: NIKKOL PUREPHOS α, manufactured by Nikko Chemicals Co., Ltd.) and others, polyols such as glycerin, DPG, and others, cationic polymers such as polyquaternium-10, guar hydroxypropyltrimonium chloride, and others, pH buffers such as citric acid, arginine, and others, antiseptics such as sodium benzoate, phenoxyethanol, and others, hydrolyzed proteins, peptides, amino acids, vitamins, UV absorbers, and others.

Dosage forms of the hair treatment agent in the present invention are not particularly limited. In an embodiment, the hair treatment agent is preferably a hair treatment (including those of a leave-in type), a hair conditioner, a hair mask, or a hair pack.

The product of the present invention is described in more detail by giving examples below, but the present invention is not limited thereto.

### EXAMPLES

### 1. Method for Preparing Hair Treatment Agent

The hair treatment agents shown in Table 1 containing the components of the product of the present invention were prepared. In a preparation method, phase A was added to and emulsified with phase B at 80° C., and they were cooled slowly at room temperature and mixed until they became uniform to obtain the products of the present invention of examples 1 to 4 and the comparative products of comparative examples 1 to 6. The numerical values for the components in the table are expressed as mass %. The prepared hair treatment agents all took the form of cream.

### 2. Evaluation of Moistness

How each sample prepared in the above 1. felt when used was evaluated using the same. Six in-house participants made sensory evaluations on the basis of the following evaluation criteria. Table 1 shows the results of averaging the sensory evaluations.

### <<Evaluation Criteria>>

A: good
B: there was not much perceivable difference whether the sample was formulated therewith or not
C: poor

### 3. Evaluation of Slipperiness

The slipperiness of each sample prepared in the above 1. was evaluated using the same. The evaluation was performed by measuring the coefficient of dynamic friction (MIU) using a friction tester (KES-SE, KATO TECH CO., LTD.) under the conditions of a load of 25 g and a movement speed of 1 mm/sec. The evaluation criteria are as follows.

### <<Evaluation Criteria>>

AA: 0.2 or more lower than the not-formulated formula
A: 0.1 or more and less than 0.2 lower than the not-formulated formula
B: 0 or more and less than 0.1 lower than the not-formulated formula
C: higher than the not-formulated formula

### 4. Evaluation of Water Repellency

The water repellency of each sample prepared in the above 1. was evaluated using the same. The evaluation was performed using a contact angle meter (DropMaster DM-501, manufactured by Kyowa Interface Science Co., Ltd.) The evaluation criteria are as follows.

### <<Evaluation Criteria>>

AA: 20° or more higher than the not-formulated formula
A: 10° or more and less than 20° higher than the not-formulated formula
B: 0° or more and less than 10° higher than the not-formulated formula
C: lower than the not-formulated formula

### Evaluation Method

A bundle of hair that had undergone a general bleaching process was washed with a very common shampoo and rinsed, and thereafter subjected to a treatment process using each hair treatment agent shown in Table 1 and rinsed with water. After drying with a towel and then drying with a hair dryer, the evaluations described in the above 2. to 4. were performed.

### 5. The Esterification Rates of Component (a) Used in the Examples and the Polyglyceryl Poly (Hydroxystearate) Used as a Comparative Product are Shown Below

Polyglyceryl-10 pentahydroxystearate (esterification rate 42%)
Polyglyceryl-10 heptahydroxystearate (esterification rate 58%)
Polyglyceryl-6 pentahydroxystearate (esterification rate 63%): Polyglyceryl-3 monohydroxystearate (esterification rate 20%)
Polyglyceryl-6 polyhydroxystearate (esterification rate 13%)

Further, polyglyceryl-10 pentaoleate (esterification rate 42%) was also used as a comparative material.

### 6. Result

As shown in Table 1, the products of the present invention formulated with a predetermined polyglycerin hydroxystearate ester as component (a) were effective at improving the moistness, slipperiness, and water repellency of hair compared to a product not formulated with the same (examples 1 to 4 and comparative example 5). On the other hand, the comparative products formulated with polyglycerin esterified using oleic acid (comparative examples 1 and 2) increased hydrophilicity of the surface of hair as mentioned earlier and thus did not attain the effect of water repellency. Further, in comparative examples 3 and 4 using a polymer of hydroxystearic acid like polyglyceryl-6 polyhydroxystearate, the slipperiness or water repellency was inferior. Further, when the degree of polymerization of polyglycerin was low as in polyglyceryl-3 monohydroxystearate, the slipperiness or water repellency was also inferior, as seen in comparative example 6.

TABLE 1

| | | Not- | | | | | | | | | | |
|---|---|---|---|---|---|---|---|---|---|---|---|---|
| | | formulated | | | | | Comparative product | | | | | |
| | | Not- | Product of present invention | | | | Compar- | Compar- | Compar- | Compar- | Compar- | Compar- |
| | | formulated | Exam- | Exam- | Exam- | Exam- | ative | ative | ative | ative | ative | ative |
| Component | | formula | ple 1 | ple 2 | ple 3 | ple 4 | example 1 | example 2 | example 3 | example 4 | example 5 | example 6 |
| A | Polyglyceryl-10 | | 0.5 | 2 | | | | | | | | |
| | pentahydroxystearate | | | | | | | | | | | |
| | Polyglyceryl-10 | | | | 0.25 | | | | | | | |
| | heptahydroxystearate | | | | | | | | | | | |
| | Polyglyceryl-6 | | | | | 0.5 | | | | | | |
| | pentahydroxystearate | | | | | | | | | | | |
| | Polyglyceryl-3 | | | | | | | | | | | 0.5 |
| | monohydroxystearate | | | | | | | | | | | |
| | Polyglyceryl-10 | | | | | | 0.5 | 0.25 | | | | |
| | pentaoleate | | | | | | | | | | | |
| | Polyglyceryl-6 | | | | | | | | 0.5 | 2 | | |
| | polyhydroxystearate | | | | | | | | | | | |
| | 2-perfluoroalkyl | | | | | | | | | | 2 | |
| | alcohol | | | | | | | | | | | |
| | Cetostearyl alcohol | 6 | 6 | 6 | 11 | 6 | 6 | 11 | 6 | 6 | 6 | 6 |
| | Glyceryl stearate | 0.5 | 0.5 | 0.5 | 0.5 | 0.5 | 0.5 | 0.5 | 0.5 | 0.5 | 0.5 | 0.5 |
| | Ethylhexyl | 2 | 2 | 2 | 2 | 2 | 2 | 2 | 2 | 2 | 2 | 2 |
| | palmitate | | | | | | | | | | | |
| B | Steartrimonium | 2 | 2 | 2 | 5 | 2 | 2 | 5 | 2 | 2 | 2 | 2 |
| | chloride | | | | | | | | | | | |
| | Ethanol | 2 | 2 | 2 | 2 | 2 | 2 | 2 | 2 | 2 | 2 | 2 |
| | Phenoxyethanol | 0.5 | 0.5 | 0.5 | 0.5 | 0.5 | 0.5 | 0.5 | 0.5 | 0.5 | 0.5 | 0.5 |
| | PG | 3 | 3 | 3 | 3 | 3 | 3 | 3 | 3 | 3 | 3 | 3 |
| | pH adjuster | Appro- | Appro- | Appro- | Appro- | Appro- | Appro- | Appro- | Appro- | Appro- | Appro- | Appro- |
| | | priate | priate | priate | priate | priate | priate | priate | priate | priate | priate | priate |
| | | amount | amount | amount | amount | amount | amount | amount | amount | amount | amount | amount |
| | Purified water | All the | All the | All the | All the | All the | All the | All the | All the | All the | All the | All the |
| | | rest | rest | rest | rest | rest | rest | rest | rest | rest | rest | rest |

| | | | | | | | | | | | |
|---|---|---|---|---|---|---|---|---|---|---|---|
| Total | 100 | 100 | 100 | 100 | 100 | 100 | 100 | 100 | 100 | 100 | 100 |
| Moistness of hair | — | A | A | A | A | A | A | B | B | C | A |
| Slipperiness of hair | — | AA | A | AA | A | A | AA | B | C | A | B |
| Water repellency of hair | — | AA | AA | AA | A | C | C | C | C | A | B |

The applications of the hair treatment agent where the effect of the present invention is perceivable are illustrated below. The formulation amounts are expressed as mass %. It was able to be confirmed by the evaluation methods explained in the above 2. to 4. that the applied products of the hair treatment agent obtained in examples 5 to 8 all attained excellent moistness, slipperiness, and water repellency.

### Example 5: Hair Conditioner

| | | | |
|---|---|---|---|
| Phase A: | Polyglyceryl-6 | 0.5 | (mass %) |
| | pentahydroxystearate | | |
| | Squalane | 5.0 | |
| | Ethylhexyl palmitate | 2.0 | |
| | Cetanol | 5.5 | |
| Phase B: | Steartrimonium chloride | 2.5 | |
| | Ethanol | 2.5 | |
| | Propanediol | 5.0 | |
| | Glycerin | 5.0 | |
| | Citric acid | Appropriate amount | |
| | Sodium citrate | Appropriate amount | |
| | Antiseptic | Appropriate amount | |
| | Water | All the rest | |
| Total | | 100.0 | |

Preparation method: the same as 1. Method for preparing hair treatment agent described in Examples.
Result: good moistness, slipperiness, and water repellency were attained.

### Example 6: Hair Mask

| | | | |
|---|---|---|---|
| Phase A: | Polyglyceryl-6 | 1.0 | (mass %) |
| | heptahydroxystearate | | |
| | Dimethicone | 8.0 | |
| | Amodimethicone | 2.0 | |
| | Cetostearyl alcohol | 6.5 | |
| Phase B: | Behenamidopropyl | 3.0 | |
| | dimethylamine | | |
| | Lactic acid | 1.0 | |
| | Propanediol | 5.0 | |
| | Glycerin | 5.0 | |
| | Dipropylene glycol | 3.0 | |
| | Hydrolyzed keratin | Appropriate amount | |
| | Zinc glycinate | Appropriate amount | |
| | Aspartic acid | Appropriate amount | |
| | Antiseptic | Appropriate amount | |
| | Water | All the rest | |
| Total | | 100.0 | |

Preparation method: the same as 1. Method for preparing hair treatment agent described in Examples.
Result: good moistness, slipperiness, and water repellency were attained.

### Example 7: Hair Treatment

| | | | |
|---|---|---|---|
| Phase A: | Polyglyceryl-10 | 1.0 | (mass %) |
| | trihydroxystearate | | |
| | Squalane | 5.0 | |
| | Glyceryl | 4.0 | |
| | tri(caprylate/caprate) | | |
| | Amodimethicone | 2.0 | |
| | Cetostearyl alcohol | 3.5 | |
| | Behenyl alcohol | 3.0 | |
| Phase B: | Disteardimonium chloride | 2.0 | |
| | PPG-24-glycereth-24 | 1.0 | |
| | Isopropyl alcohol | 1.0 | |
| | Glycerin | 5.0 | |
| | Dipropylene glycol | 3.0 | |
| | Hydrolyzed keratin | Appropriate amount | |
| | Antiseptic | Appropriate amount | |
| | Water | All the rest | |
| Total | | 100.0 | |

Preparation method: the same as 1. Method for preparing hair treatment agent described in Examples.
Result: good moistness, slipperiness, and water repellency were attained.

### Example 8: Hair Treatment

| | | | |
|---|---|---|---|
| Phase A: | Polyglyceryl-10 | 1.0 | (mass %) |
| | pentahydroxystearate | | |
| | Cyclopentasiloxane | 10.0 | |
| | Cetanol | 10.0 | |
| Phase B: | Cetyl phosphate | 0.5 | |
| | Arginine | 0.3 | |
| | Citric acid | 1.0 | |
| | Glycerin | 5.0 | |
| | Dipropylene glycol | 3.0 | |
| | pH adjuster | Appropriate amount | |
| | Antiseptic | Appropriate amount | |
| | Water | All the rest | |
| Total | | 100.0 | |

Preparation method: the same as 1. Method for preparing hair treatment agent described in Examples.
Result: good moistness, slipperiness, and water repellency were attained.

### INDUSTRIAL APPLICABILITY

The hair treatment agent of the present invention can be used for imparting sufficient moistness, slipperiness, and water repellency to hair damaged by chemical processes and made to be hydrophilic.

1. A hair treatment agent comprising a component (a) which is a polyglycerin hydroxystearate ester having 0.5 to 10.0 mol of hydroxystearic acid relative to 1 mol of polyglycerin in which an average degree of polymerization of glycerin is 5 or more.

2. The hair treatment agent according to claim 1, wherein a formulation amount of the polyglycerin hydroxystearate ester of the component (a) is 0.1 to 5.0 mass % of a total mass of the hair treatment agent.

3. The hair treatment agent according to claim 1, wherein the hair treatment agent is further formulated with a component (b) which is one or more selected from the group consisting of a mono (long-chain alkyl) quaternary ammonium salt, a di (long-chain alkyl) quaternary ammonium salt, and a mono (long-chain alkyl) amine.

4. The hair treatment agent according to claim 1, wherein [number of esterified hydroxyl groups of polyglycerin]/[total number of hydroxyl groups of polyglycerin] of the polyglycerin hydroxystearate ester of the component (a) is 20% to 90%.

5. The hair treatment agent according to claim 3, wherein a formulation amount of the component (b) is 0.1 to 5.0 mass % of a total mass of the hair treatment agent.

6. The hair treatment agent according to claim 3, wherein a mass ratio expressed as [content of component (b)]/[content of component (a)] is 0.5 to 30.

7. The hair treatment agent according to claim 1, wherein the polyglycerin hydroxystearate ester of the component (a) is selected from the group consisting of polyglyceryl-10 decahydroxystearate, polyglyceryl-10 heptahydroxystearate, polyglyceryl-10 pentahydroxystearate, polyglyceryl-6 pentahydroxystearate, and a combination of two or more of these.

8. The hair treatment agent according to claim 3, wherein the component (b) is one or more selected from the group consisting of behentrimonium chloride, steartrimonium chloride, cetrimonium chloride, stearamidopropyl dimethylamine, and behenamidopropyl dimethylamine.

9. The hair treatment agent according to claim 1, wherein the hair treatment agent is a hair treatment, a hair conditioner, a hair mask, or a hair pack.
